(19) United States
(12) Patent Application Publication
(10) Pub. No.: US 2015/0073156 A1
(43) Pub. Date: Mar. 12, 2015

(54) Production Method of a-Methylene Lactone

(71) Applicant: Cheil Industries Inc., Gumi-si (KR)

(72) Inventors: Myung Ryul Lee, Uiwang-si (KR); Sun Dae Kim, Uiwang-si (KR); In Sik Jeon, Uiwang-si (KR); Won Gi Lee, Uiwang-si (KR); Sung Hee Ahn, Uiwang-si (KR); Sang Hyun Hong, Uiwang-si (KR)

(21) Appl. No.: 14/368,663

(22) PCT Filed: Jul. 20, 2012
(86) PCT No.: PCT/KR2012/005821

§ 371 Date: Oct. 8, 2014

(30) Foreign Application Priority Data
Jan. 13, 2012 (KR) 10-2012-0004314

(51) Int. Cl. C07D 307/58

(52) U.S. Cl. CPC C07D 307/58
USPC 549/326; 549/295

(57) ABSTRACT

The present invention relates to a production method of α-methylene lactone which comprises the following steps: (A) a step of producing an enolate intermediate by making lactone react with alkyl formate under the presence of an alkoxide base; and (B) making the enolate intermediate react with paraformaldehyde. The production method of the present invention is capable of reducing the process time, improving yield, and minimizing the contamination of a reactor.

### FIELD OF THE INVENTION

The present invention relates to a production method of α-methylene lactone. More specifically, the present invention relates to a production method of α-methylene lactone in high yield including a step of producing an enolate intermediate from lactone, and a step of making the enolate intermediate react with paraformaldehyde.

### BACKGROUND OF THE INVENTION

α-Methylene lactone has been a major subject for a crucial synthesis research so far. Especially α-methylene-γ-butyrolactone among the α-methylene lactones is a γ-butyrolactone compound with an exomethylene group; has an antitumor activity, antibacterial activity, antifungal activity, and other biological activities; and is a compound important to pharmaceutical industries. A copolymer of the α-methylene-γ-butyrolactone has excellent heat resistance and transparency, high refractive index, and excellent thermal and optical stability; and is considered as a co-monomer with an acryl based or styrene based monomer for proceeding a research of the effective synthesis of the copolymer.

Japanese disclosed patent 2001-247560 provides a production method of the α-methylene-γ-butyrolactone by making γ-butyrolactone react with methanol under the presence of a manganese/magnesium oxide (Mn/MgO) catalyst. However, this method is hard to be commercially used due to the low conversion ratio when a user uses the methanol after converting in a reactor as a source for formaldehyde.

Other production method of the α-methylene-γ-butyrolactone is possible by making homopropargyl alcohol react with carbon monoxide under the presence of a nickel or palladium catalyst. However, when the production method uses a nickel catalyst, said production method is also hard to be applied to an industrial production due to the low yield and generation of side reactions such as acetylene polymerization and double bonding transfer. Furthermore, when using a palladium catalyst, the yield can be improved to be greater than 90% but the recovery of the catalyst is hard because of using a homogeneous catalyst (J. Am. Chem. Soc. 1981, volume 103, p. 7520). In said method, a process which is capable of reusing the catalyst has been reported by making a palladium homogeneous catalyst react under the presence of ionic liquid, however the homopropargyl alcohol is expensive and therefore the method has low economic feasibility (Tetrahedron Lett. 2002, volume 43, p. 753).

Additionally, a production method of a cyclic unsaturated compound is disclosed by using acrylic acid, ethylene, and a palladium homogeneous and non-homogeneous catalyst (Japanese registration patent 4642116 and US Publication 2009-0299009 A1). However said method has the yield greater than 60% when using the homogeneous catalyst but is incapable of recovering the catalyst, and has the yield less than 15% when using the non-homogeneous catalyst.

Previously described exiting methods have advantages of a one-step process, but the lifetime of the catalyst is short due to the high temperature and high pressure reaction condition, and expensive noble metal catalysts or homopropargyl alcohol are used for the reaction; therefore is incapable of being used for industrial production.

Additionally, the α-methylene-γ-butyrolactone is capable of being produced by a two-step process instead of the one-step process. The existing two-step process comprises the following steps: a first step of producing enolate which is an intermediate by making the γ-butyrolactone react with ethyl formate under the presence of NaH; and a second step of making the enolate react with paraformaldehyde to obtain the α-methylene-γ-butyrolactone. However, in the first step of producing enolate salt, solid phase salt in a solvent is eluted, therefore a filtering process is more important than anything. But no reports have been filed regarding the particle size of the enolate and the filtering speed. Also in the second step, the excessive amount of paraformaldehyde is used and the paraformaldehyde has a possibility of contaminating a reactor, thereby being unsuitable for a process of mass production.

For solving problems of existing technique, the present inventor used a different catalyst and changed the reaction condition for developing a method of the present invention of producing the α-methylene lactone in high yield.

### DETAILED DESCRIPTION OF THE INVENTION

### Technical Subject

The object of the present invention relates to a production method of α-methylene lactone with excellent process efficiency.

Another object of the present invention relates to a production method of α-methylene lactone in high yield.

Another object of the present invention relates to a production method of α-methylene lactone which can minimize the contamination of a reactor.

The aforementioned and other objects of the present invention will be achieved by the present invention as described below.

### TECHNICAL SOLUTION

The present invention provides a production method of α-methylene lactone which comprises the following steps: (A) a step of producing an enolate intermediate by making lactone react with alkyl formate under the presence of an alkoxide base; and (B) making the enolate intermediate react with paraformaldehyde.

The enolate intermediate is capable of being produced by the reaction denoted by reaction formula 1, and α-methylene-γ-butyrolactone is capable of being produced by the reaction denoted by reaction formula 2.

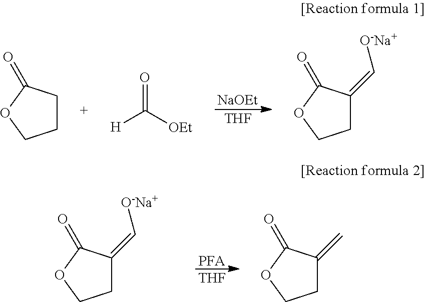

The step (A) for producing the enolate intermediate preferably uses 1 to 1.5 equivalent of alkyl formate, and 0.7 to 1.5 equivalent of alkoxide base based on 1 equivalent of lactone. The lactone and a solvent are capable of being used in a weight ratio of 1:5 to 1:10 in the step (A) for producing the enolate intermediate.

The step (A) for producing the enolate intermediate is preferable when the reaction is performed in a reactor with the size of 1 to 100 L, at a temperature of 10 to 40° C., and in a stirring speed of 50 to 150 rpm; and is preferable when the reaction is performed at 10 to 40° C. and in a stirring speed of 30 to 80 rpm for the reactor with the size exceeding 100 L.

The step (B) for producing α-methylene lactone preferably uses 1 to 4 equivalent of paraformaldehyde based on 1 equivalent of the enolate intermediate. The enolate intermediate and a solvent in the step (B) for producing the α-methylene lactone are used in a weight ratio of 1:7 to 1:15. The reaction temperature for the step (B) for producing the α-methylene lactone is preferably 10 to 40° C.

### SIMPLE DESCRIPTION OF DRAWINGS

FIG. 1 is a figure representing the ¹H-NMR peak of an enolate intermediate which is an intermediate of the present invention.

FIG. 2 is a figure representing the ¹H-NMR peak of α-methylene lactone which is a final product of the present invention.

FIG. 3 is a figure representing the particle size analysis result of example 1.

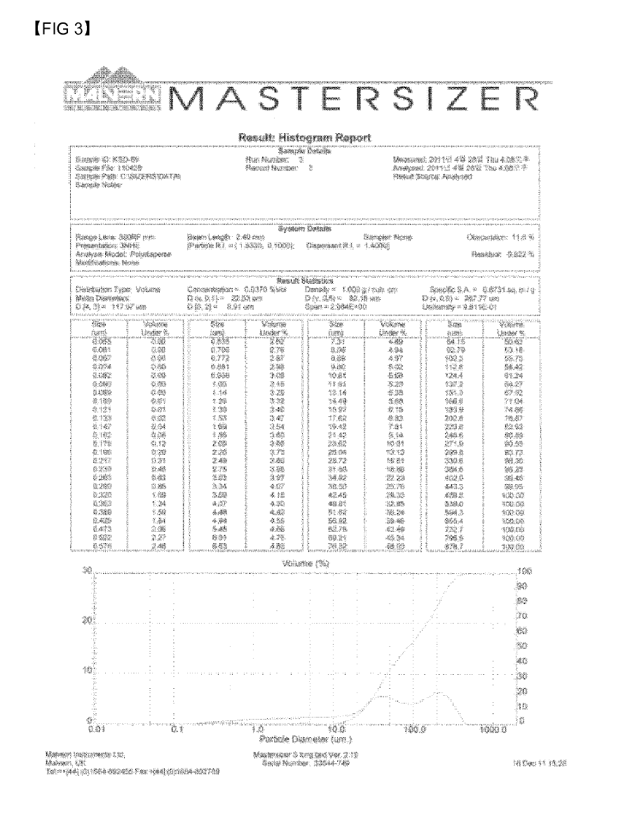

FIG. 4 is a figure representing the particle size analysis result of example 2.

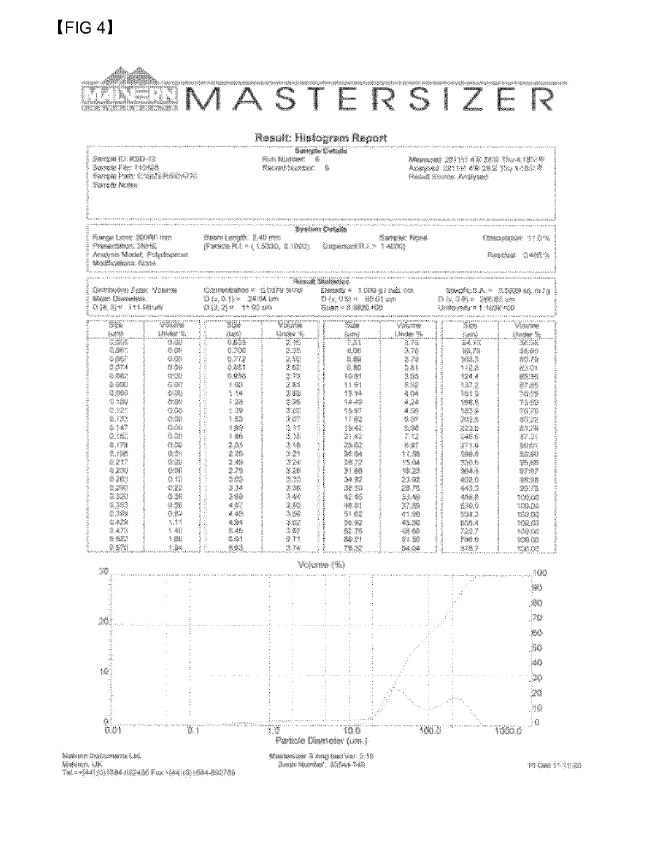

FIG. 5 is a figure representing the particle size analysis result of example 3.

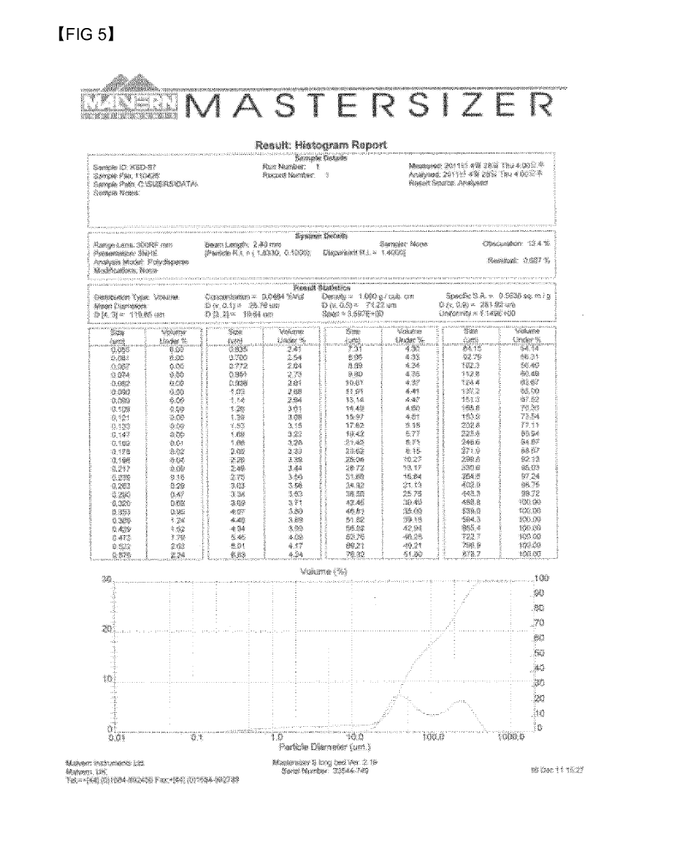

FIG. 6 is a figure representing the particle size analysis result of comparative example 1.

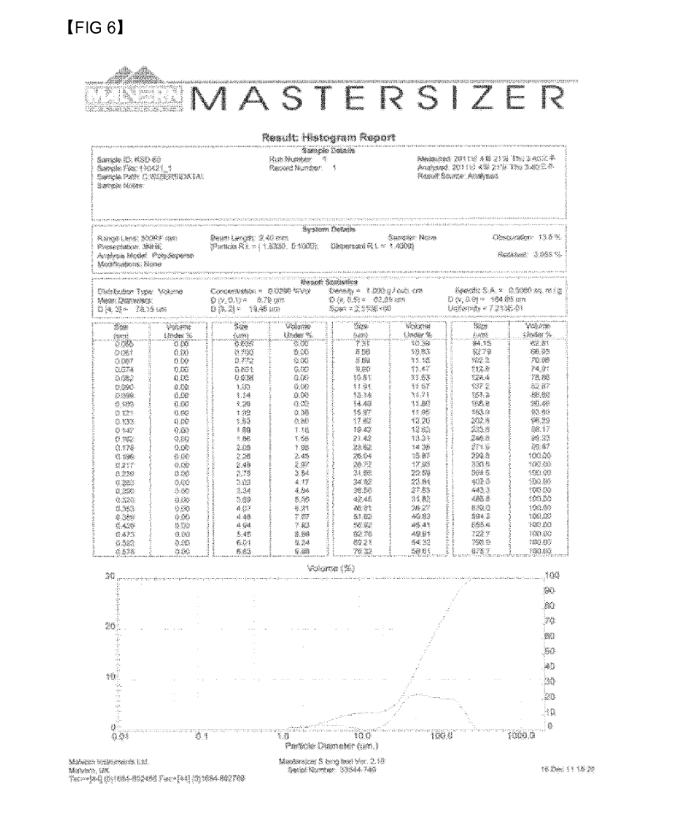

### OPTIMAL EMBODIMENT OF THE PRESENT INVENTION

The present invention relates to a production method of α-methylene lactone, which comprises a step of producing an enolate intermediate from lactone, and a step of making the enolate intermediate react with paraformaldehyde to obtain the α-methylene lactone in high yield.

The present invention relates to a production method of the α-methylene lactone which comprises the following steps: (A) a step of producing enolate intermediate by making the lactone react with alkyl formate under the presence of an alkoxide base; and (B) making the enolate intermediate react with paraformaldehyde. The α-methylene lactone of the present invention is capable of being produced by the reaction denoted by reaction formula 3 as an embodiment of the present invention

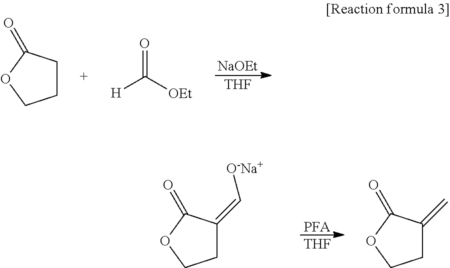

The present invention is specifically described below.

### (A) Enolate Intermediate Production Step

In the present invention, an enolate intermediate is produced by making lactone react with alkyl formate under the presence of an alkoxide base. Lactone with five rings or six rings is capable of being used, and preferably γ-butyrolactone is used as the lactone. The final product is α-methylene-γ-butyrolactone when using the γ-butyrolactone.

In the present invention, the alkoxide base is used as a base for removing α-hydrogen from the lactone, and preferably sodium ethoxide (NaOEt) is used as the alkoxide base.

Additionally, the present invention uses the alkyl formate as a precursor for introducing a methylene group to the α-location of the lactone, and preferably ethyl formate is used as the alkyl formate.

According to an embodiment of the present invention, the enolate intermediate is produced by the reaction denoted by reaction formula 1.

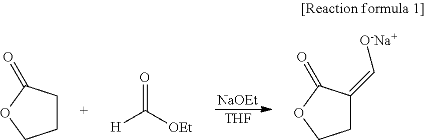

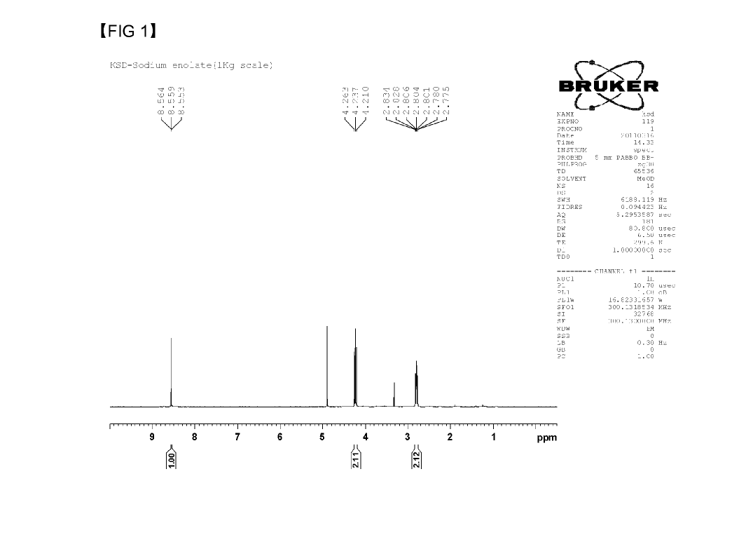

The reaction denoted by the reaction formula 1 is a reaction where the lactone is the γ-butyrolactone, the alkyl formate is the ethyl formate, the alkoxide base is the sodium ethoxide, and a solvent is tetrahydrofuran (THF). And the product of the reaction is α-formyl-γ-butyrolactone sodium salt which can be confirmed by analyzing the ¹H-NMR peak of FIG. 1.

The enolate intermediate produced from the present step is solid phase salt, and the improvement of yield by reducing the filtration time regardless of the size of filtering paper is important by adjusting the size of generated particles.

In the present invention, preferable usage amount of the alkyl formate is 1-2 equivalent and the preferable usage amount of the alkoxide base is 0.7-1.5 equivalent when using 1 equivalent of the lactone. The desired particle size of the enolate intermediate of the present invention is impossible to obtain when the present invention exceeds said equivalent rate.

The solvents for the present invention are capable of being selected from the group consisting of: alcohols including methanol, ethanol, n-butanol; esters including tetrahydrofuran (THF) and dioxane; aromatic compounds including toluene and xylene; and polar solvents.

In the enolate intermediate production step (A), the lactone and the solvent are preferably used in a weight ratio of 1:5 to 1:10. The size of the enolate intermediate decreases when the solvent is used less than said range, the filtering paper with the big size is incapable of being used, and the small sized filtering paper is capable of increasing filtration time.

In the present invention, the stirring speed of a reaction result is capable of varying by the capacity of a reactor. 50 to 150 rpm of stirring speed is preferable when a small scale reactor with the size of 1 to 100 L is used, and more preferably 80 to 120 rpm of stirring speed is possible. 30 to 80 rpm of stirring speed is possible when a big scale reactor with the size exceeding 100 L is used, more preferably 40 to 60 rpm of stirring speed is possible. The enolate intermediate in the desired size is impossible to obtain when the stirring speed exceeds said range.

Also the reaction temperature is at 10 to 40° C., which is close to room temperature, and the reaction does not require a high temperature and high pressure condition.

The enolate intermediate produced by the reaction preferably have less than 5% of volume ratio of 1 to 10 μm particles. Dense filtering paper should be used when the volume ratio exceeds 5%, and accordingly the filtration time increases.

The synthesis yield of the enolate intermediate obtained by the reaction and filtration process is greater than 75%.

### (B) α-Methylene Lactone Production Step

In the present invention, the α-methylene lactone is capable of being produced by making the produced enolate intermediate react with paraformaldehyde, and 1 to 5 equivalent of paraformaldehyde per 1 equivalent of enolate intermediate is preferable.

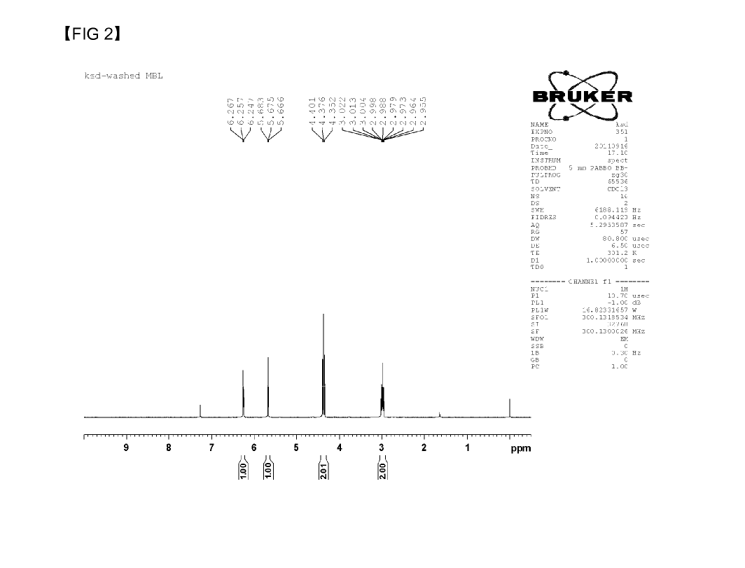

In the production of the α-methylene lactone, when using γ-butyrolactone as a lactone component in the enolate intermediate production step (A), α-methylene-γ-butyrolactone denoted by chemical formula 1 can be obtained, and which can be confirmed by analyzing the ¹H-NMR peak of FIG. 2.

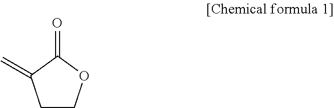

In the present invention, the α-methylene lactone is obtained by making the produced enolate intermediate react with the paraformaldehyde, and the α-methylene-γ-butyrolactone is produced by the reaction formula 2.

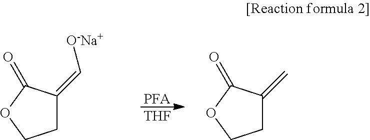

In the α-methylene lactone production step (B), 1 to 5 equivalent of paraformaldehyde per 1 equivalent of the enolate intermediate is preferable.

The enolate intermediate and a solvent of the α-methylene lactone production step (B) are preferably used in a weight ratio of 1:7 to 1:15. When the enolate intermediate and the solvent are used less than said range, the heat generation control is hard and the yield is decreased.

Additionally the α-methylene lactone production step (B) is capable of being performed at 10 to 40° C., preferably at 15 to 20° C. By making the reflux condition of a tetrahydrofuran solvent close to room temperature, the contamination of a reaction by the paraformaldehyde is capable of being minimized, and the energy and power consumption is capable of being reduced.

The α-methylene lactone obtained by the reaction and filtration is synthesized in yield greater than 75%.

Preferable embodiments of the present invention are disclosed below. However the embodiments are for suggesting the preferable examples of the present invention, not for limiting the present invention.

### Embodiment of the Present Invention

### Examples

### Examples 1-4 and Comparative Examples 1-2

### The Production of an Enolate Intermediate

### Example 1

Sodium ethoxide (395 g, 5.81 mol) and 3.5 L of tetrahydrofuran (THF) as a solvent are inserted into a 5 L reactor, and the mixture is stirred in 115 rpm while maintaining the temperature of the reactor at 17° C. Ethyl formate (645 g, 8.72 mol) is rapidly inserted into the reactor, and γ-butyrolactone (500 g, 5.81 mol) is slowly dropped into the reactor for 1 hour and 30 minutes. The internal temperature of the reactor does not exceed 30° C. when the γ-butyrolactone is dropped into the reactor, and the internal temperature of the reactor is maintained at 17° C. for 20 hours after the dropping of the γ-butyrolactone while stirring. Precipitated compound after the finish of the reaction is filtered by using a 3 μm paper filter, and is washed with THF. The filtered compound is dried in a 60° C. vacuum oven, and α-formyl-γ-butyrolactone sodium salt is synthesized.

### Example 2

α-formyl-γ-butyrolactone sodium salt is synthesized by the same method of the example 1, except for stirring a mixture in the speed of 115 rpm and using a 1 μm paper filter for filtering.

### Example 3

α-formyl-γ-butyrolactone sodium salt is synthesized by the same method of the example 1, except for stirring a mixture in the speed of 115 rpm and using a 10 μm paper filter for filtering.

### Example 4

Sodium ethoxide (16.1 kg, 0.24 kmol) and 143 L of tetrahydrofuran (THF) as a solvent are inserted into a 630 L reactor, and the mixture is stirred in 50 rpm while maintaining the temperature of the reactor at 17° C. Ethyl formate (26.3 kg, 0.35 kmol) is rapidly inserted into the reactor, and γ-butyrolactone (20.3 kg, 0.24 kmol) is slowly dropped into the reactor for 1 hour and 30 minutes using a metering pump. The internal temperature of the reactor does not exceed 30° C. when the γ-butyrolactone is dropped into the reactor, and the internal temperature of the reactor is maintained at 17° C. for 20 hours after the dropping of the γ-butyrolactone while stirring. Precipitated compound after the finish of the reaction is filtered by using a Nutsche filter and a 10 μm paper filter, and is washed with THF. The filtered compound is dried in a 60° C. vacuum oven, and α-formyl-γ-butyrolactone sodium salt is synthesized.

### Comparative Example 1

α-formyl-γ-butyrolactone sodium salt is synthesized by the same method of the example 1, except for stirring a mixture in the speed of 185 rpm.

### Comparative Example 2

α-formyl-γ-butyrolactone sodium salt is synthesized by the same method of the example 4, except for stirring a mixture in the speed of 100 rpm.

The correlation of particulate volume rate based on stirring speed, filtration time based on the size of the filtering paper, and yield are arranged in table 1. The particulate volume rate means the rate of the volume of 1 to 10.81 μm particles among the entire particles.

TABLE 1

| | Stirring | Particulate | | | |
|---|---|---|---|---|---|
| | speed | volume rate | Filtering | Filtration | |
| | (rpm) | (%) | paper (μm) | time (min) | Yield |
| Example 1 | 115 | 1.92 | 3 | 10 | 90 |
| Example 2 | 115 | 1.04 | 1 | 12 | 90 |
| Example 3 | 115 | 1.49 | 10 | 9 | 90 |
| Example 4 | 50 | 1.72 | 10 | 120 | 85 |
| Comparative | 185 | 11.63 | 3 | 68 | 74 |
| example 1 | | | | | |
| Comparative | 100 | 10.95 | 10 | 720 | 70 |
| example 2 | | | | | |

As mentioned above on the table 1, according to the results of the examples 1 to 4, the particulate volume rate is small, the filtration time is reduced regardless of the size of the filtering paper, and the yield is improved.

However, in the comparative examples 1 and 2, the filtration time of remarkably increased and the yield is reduced by the big particulate volume rate.

### Examples 5-6 and Comparative Examples 3-4

### The Production of α-Methylene Lactone

### Example 5

α-formyl-γ-butyrolactone sodium salt (711.5 g, 5.23 mol) and 7.1 L of tetrahydrofuran (THF) as a solvent is inserted into a 10 L reactor, and the mixture is stirred in 185 rpm while maintaining the temperature of the reactor at 17° C. Paraformaldehyde (627.6 g, 20.9 mol) is rapidly inserted into the reactor after producing slurry of the paraformaldehyde in 1 L of tetrahydrofuran solvent. The mixture is stirred for 5 hours in said condition and filtered and washed by using a 10 μm paper filter, and the filtrate is concentrated and decompress-distilled to obtain α-methylene-γ-butyrolactone.

### Example 6

α-formyl-γ-butyrolactone sodium salt (27.4 kg, 0.2 kmol) and 200 L of tetrahydrofuran (THF) as a solvent is inserted into a 630 L reactor, and the mixture is stirred in 100 rpm while maintaining the temperature of the reactor at 17° C. Paraformaldehyde (24.0 kg, 0.8 kmol) is rapidly inserted into the reactor after producing slurry of the paraformaldehyde in 73.5 L of tetrahydrofuran solvent. The mixture is stirred for 5 hours in said condition and filtered and washed by using a Nutsche filter and a 10 μm paper filter, and the filtrate is concentrated and decompress-distilled to obtain α-methylene-γ-butyrolactone.

### Comparative Example 3

α-methylene-γ-butyrolactone is synthesized by the same method of the example 5, except for the reaction at 80° C.

### Comparative Example 4

α-methylene-γ-butyrolactone is synthesized by the same method of the example 6, except for the reaction at 80° C.

The contamination of a reactor and yield of examples 5 to 6 and comparative example 3 to 4 are arranged in table 2. The contamination of the reactor is visually confirmed. (Contamination: O, Non-contamination: X)

TABLE 2

| | Contamination of reactor | Yield |
|---|---|---|
| Example 5 | X | 80 |
| Example 6 | X | 85 |
| Comparative example 3 | ◯ | 55 |
| Comparative example 4 | ◯ | 62 |

As mentioned above on the table 2, according to the results of the examples 5 and 6, the reactor is not contaminated and the yield is superior.

However, in the comparative examples 3 and 4, the contamination is generated in a reflux condenser and the yield is decreased.

1. A method of producing α-methylene lactone which comprises the following steps:
(A) reacting lactone with alkyl formate in the presence of an alkoxide base to produce an enolate intermediate; and
(B) reacting the enolate intermediate with paraformaldehyde.

2. The method of producing α-methylene lactone of claim 1, which produces α-methylene-γ-butyrolactone by using γ-butyrolactone as the lactone.

3. The method of producing α-methylene lactone of claim 2, wherein the alkyl formate is ethyl formate.

4. The method of producing α-methylene lactone of claim 3, wherein the alkoxide base is sodium ethoxide.

5. The method of producing α-methylene lactone of claim 4, wherein the enolate intermediate is produced by a reaction denoted by reaction formula 1, and the α-methylene-γ-butyrolactone is produced by a reaction denoted by reaction formula 2:

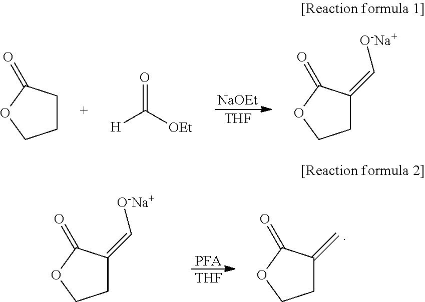

6. The method of producing α-methylene lactone of claim 1, wherein the enolate intermediate production step (A) uses 1 to 1.5 equivalent of alkyl formate, and 0.7 to 1.5 equivalent of alkoxide base per 1 equivalent of lactone.

7. The method of producing α-methylene lactone of claim 5, wherein the enolate intermediate production step (A) uses lactone and a solvent tetrahydrofuran (THF) in a weight ratio of 1:5 to 1:10.

8. The method of producing α-methylene lactone of claim 1, wherein the enolate intermediate production step (A) performs a reaction at a temperature of 10 to 40° C. at a stirring speed of 50 to 150 rpm in a reactor with a size of 1 to 100 L.

9. The method of producing α-methylene lactone of claim 1, wherein the enolate intermediate production step (A) performs a reaction at 10 to 40° C. at a stirring speed of 30 to 80 rpm in a reactor with a size exceeding 100 L.

10. The method of producing α-methylene lactone of claim 1, wherein the α-methylene lactone production step (B) uses 1 to 4 equivalent of paraformaldehyde per 1 equivalent of enolate intermediate.

11. The method of producing α-methylene lactone of claim 5, wherein the α-methylene lactone production step (B) uses the enolate intermediate and a solvent tetrahydrofuran (THF) in a weight ratio of 1:7 to 1:15.

12. The method of producing α-methylene lactone of claim 1, wherein the α-methylene lactone production step (B) performs a reaction at a temperature of 10 to 40° C.

13. The method of producing α-methylene lactone of claim 1, wherein the enolate intermediate includes less than 5% volume ratio of 1 to 10 μm enolate intermediate particles based on the total amount of enolate intermediate particles.

14. The method of producing α-methylene lactone of claim 1, wherein the synthesis yield of an enolate intermediate is greater than 75%, and the synthesis yield of the final product α-methylene lactone is greater than 75%.

15. α-Methylene lactone a methylene lactone prepared by the method of claim 1.
